(19) United States
(12) Patent Application Publication
(10) Pub. No.: US 2011/0306775 A1
(43) Pub. Date: Dec. 15, 2011

(54) SYNTHESIS AND BIOLOGICAL EVALUATION OF 2',5'-DIMETHOXYCHALCONE DERIVATIVES AS MICROTUBULE-TARGETED ANTICANCER AGENTS

(75) Inventors: Chun-Nan Lin, Kaohsiung City (TW); Huang-Yao Tu, Kaohsiung City (TW); A-Mei Huang, Kaohsiung City (TW); Tzyh-Chyuan Hoar, Kaohsiung City (TW); Shyh-Chyun Yang, Kaohsiung City (TW); Yeong-Shiau Pu, Taipei City (TW); Jan-Gowth Chang, Kaohsiung City (TW)

(73) Assignee: KAOHSIUNG MEDICAL UNIVERSITY, Kaohsiung City (TW)

(21) Appl. No.: 12/896,246

(22) Filed: Oct. 1, 2010

(30) Foreign Application Priority Data
Jun. 10, 2010 (TW) 099118967

(51) Int. Cl. C07D 207/12; C07C 233/00; C07C 69/76; C07C 59/90; C07D 333/38

(52) U.S. Cl. 548/539; 562/463; 549/71; 560/53; 564/169

(57) ABSTRACT

Disclosed are a serious of 2′,5′-dimethoxychalcone derivatives for treating cancer, wherein 2,5-dimethoxyacetophenone and methyl 4-formylbenzoate are condensed to form 4-carboxyl-2′,5′-dimethoxychalcone (compound 1), which is further reacted with alkyl halides or amines to synthesize the chalcone derivatives of compounds 2-17. In addition, 2,5-dimethoxyacetophenone is reacted with 5-formyl-2-thiophenecarboxylic acid to form compound 18 (3-(3-thiophene)carboxyl-1-(2,5-dimethoxyphenyl)prop-2-en-1-one). The synthesized 2′,5′-dimethoxychalcone derivatives can be acted as microtubule-targeted tubulin-polymerizing agents.

### FIELD OF THE INVENTION

The present invention relates to an anticancer agent and its preparation method. In particular, the present invention relates to a microtubule-targeted anticancer agent and the preparation method thereof.

### BACKGROUND OF THE INVENTION

Microtubules mainly compose of tubulins, and microtubules plays an important role in a variety of cellular process including mitosis and cell division. Various anti-mitotic agents interfering with the natural dynamics of tubulin, polymerization, and depolymerization inhibit cancer cell growth (Kim et al., 2004).

For instance, paclitaxel stabilizes microtubules by preventing the depolymerization of tubulin while the vinca alkaloids and colchicine inhibit the polymerization of tubulin. Disruption of tubulin dynamics leads to cell cycle arrest in the G2/M phase and induction of apoptosis (Kim et al., 2004). At present, a major problem of anti-mitotic agent (such as taxanes and vinca alkaloids) in clinical application is the development of drug resistance. Therefore, it needs to find and develop effective tubulin inhibitors for treating multidrug-resistant (MDR) tumors.

It is therefore attempted by the applicant to deal with the above situation encountered in the prior art.

### SUMMARY OF THE INVENTION

For developing new anti-mitotic agents, the novel chalcones are synthesized in the present invention. Chalcones are ketones having aromatic rings and have been characterized with multiple biological activities including anti-inflammatory, anti-malaria, anti-protozoal, anti-bacterial, nitric oxide inhibitory, tyrosinase inhibitory, cytotoxic, anti-leishmanial, and anti-oxidant activities (Mukherjee et al., 2001). The C-4 of ring B of the newly synthesized 2′,5′-dialkoxylchalcones is conjugated with carbonyl group (C═O) or carbamoyl group (—R—CONH₂). The experiments indicate that the compounds synthesized in the present invention have effective cytotoxicity on the urinary system-related cancer cells and show less cytotoxicity on normal cells. However, the compounds of the present invention also can be applied in cytotoxicity on other cancer cells.

The first aspect of the present invention is to provide a pharmaceutical composition for treating a cancer including a chalcone compound represented by formula I:

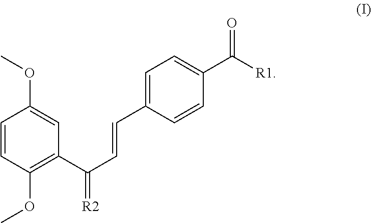

R1 and R2 have one of the following two situations, that (i) R1 is a first substituted group being one selected from a group consisting of a hydroxy group, a C₁-C₆ alkoxy group, an aromatic alkoxy group, a heterocyclic alkoxy group, an alkenyloxy group, an alkylamide group, a C₃-C₆ cycloalkylamide group, a heterocyclic amide group and an —N(CₘH₂ₘ₊₁)—CₙH₂ₙOH group with 1≦m≦6 and 1≦n≦6 when R2 is an oxygen, and (ii) R1 is a cyclopropylamide group when R2 is a cyclopropylimino group.

Preferably, the alkenyloxy group is an —OCH₂CH═CHCᵣH₂ᵣ₊₁ group with 1≦r≦6 or an —OCH₂CH═C(CₚH₂ₚ₊₁)—C_{q}H_{2q+1} group with 1≦p≦6 and 1≦q≦6.

Preferably, the alkylamide group is represented as an —NR—R′ group, and each of R and R′ is a second substituted group being one selected from a group consisting of a hydrogen, a C₁-C₆ saturated hydrocarbon group and a C₁-C₆ unsaturated hydrocarbon group.

The second aspect of the present invention is to provide a pharmaceutical composition for treating a cancer including a chalcone compound represented by formula II:

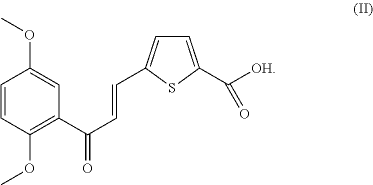

The chalcone compound is synthesized by reacting a 2,5-dimethoxyacetophenone with a 5-formyl-2-thiophenecarboxylic acid.

The third aspect of the present invention is to provide a preparation method of a pharmaceutical composition for treating a cancer, and the method includes a step of: (a) reacting 2,5-dimethoxyacetophenone with a methyl 4-formylbenzoate to obtain a 4-carboxyl-2′,5′-dimethoxychalcone.

Preferably, the step (a) is performed in an alkaline solution and then is neutralized in a first acidic solution. The alkaline solution is a potassium hydroxide solution, and the first acidic solution is a hydrochloride solution.

Preferably, the method further includes a step of: (b1) reacting the 4-carboxyl-2′,5′-dimethoxychalcone with an alkyl halide to obtain a first compound represented by the aforementioned formula I, wherein R1 is a first substituted group being one selected from a group consisting of a hydroxy group, a C₁-C₆ alkoxy group, an aromatic alkoxy group, a heterocyclic alkoxy group and an alkenyloxy group when R2 is an oxygen.

Preferably, the alkenyloxy group is an —OCH₂CH═CHCᵣH₂ᵣ₊₁ group with 1≦r≦6 or an —OCH₂CH═C(CₚH₂ₚ₊₁)—C_{q}H_{2q+1} group with 1≦p≦6 and 1≦q≦6.

Preferably, the step (b1) further includes: (b11) reacting the 4-carboxyl-2′,5′-dimethoxychalcone and the alkyl halide with a potassium carbonate to obtain a first reaction mixture; (b12) concentrating the first reaction mixture; (b13) neutralizing the first reaction mixture with a second acidic solution; and (b14) extracting the first reaction mixture with a first dichloromethane to obtain the first compound. The second acidic solution is a hydrochloride solution.

Preferably, the method further includes a step of: (b2) reacting 4-carboxyl-2′,5′-dimethoxychalcone with an amine to obtain a second compound represented by the aforementioned formula I, wherein R1 is a second substituted group being one selected from a group consisting of an alkylamide group, a C₃-C₆ cycloalkylamide group, a heterocyclic amide group and a —N(CₘH₂ₘ₊₁)—C₂H₂ₙOH group with 1≦m≦6 and 1≦n≦6 when R2 is an oxygen. The alkylamide group is an —NR—R′ group, and each of R and R′ is a third substituted group being one selected from a group consisting of a hydrogen, a C₁-C₆ saturated hydrocarbon group and a C₁-C₆ unsaturated hydrocarbon group.

Preferably, the step (b2) further includes: (b21) dissolving 4-carboxyl-2′,5′-dimethoxychalcone, hydroxybenzotriazole (HOBt) and 1-(3-dimethylaminopropyl)-3-ethylcarbodiimide hydrochloride (EDCl) in a dichloromethane to form a second mixture; (b22) reacting the second mixture with the amine to obtain a second reaction mixture; (b23) concentrating the second reaction mixture; and (b24) crystallizing the second reaction mixture with an ethyl acetate to obtain the second compound.

The fourth aspect of the present invention is to provide a preparation method of a pharmaceutical composition for treating a cancer, and the method includes a step of reacting a 2,5-dimethoxyacetophenone with a 5-formyl-2-thiophenecarboxylic acid to obtain a compound represented by the aforementioned formula II.

Preferably, the 2,5-dimethoxyacetophenone and the 5-formyl-2-thiophenecarboxylic acid are reacted in an alkaline solution and then neutralized in an acidic solution. The alkaline solution is a potassium hydroxide solution, and the acidic solution is a hydrochloride solution.

The above objectives and advantages of the present invention will become more readily apparent to those ordinarily skilled in the art after reviewing the following detailed descriptions and accompanying drawings.

### BRIEF DESCRIPTION OF THE DRAWINGS

FIG. 1 depicts the cytotoxicities of compound 15 against NTUB1, PC3, A549 and SV-HUC1 cells.

FIG. 2 depicts the cell cycle distribution of NTUB1 cells for 24 hours with compounds 11 and 15 in different concentrations.

FIG. 3 depicts the cell cycle distribution of PC3 cells treated with compounds 11 and 15 in different concentrations.

FIG. 4 depicts immunoblotting of α-tubulin in NTU cells.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

### EXPERIMENTAL METHODS

I. Preparation of compound 1 to 18:

1. Preparation of compound 1 (4-carboxyl-2′,5′-dimethoxychalcone)

2,5-Dimethoxyacetophenone (450.0 mg, 2.5 mmol) and methyl 4-formylbenzoate (410.4 mg, 2.5 mmol) were dissolved in methanol (MeOH, 50 mL), and added 8% potassium hydroxide (KOH) in H₂O (50 mL). The reaction mixture was stirred at room temperature for 24 hours and neutralized with 10% hydrochloride solution (HCl, 100 mL) to form yellow precipitate. The yellow precipitate was filtered and washed with appropriate amount of water. The crude product was purified by chromatography using ethyl acetate (EtOAc)/n-hexane (2:1), and crystallized by EtOAc/n-hexane (1:4) to afford compound 1 (4-carboxyl-2′,5′-dimethoxychalcone, 491.6 mg, 63.0%) as a yellow solid. The reaction formula of compound 1 was shown as follows.

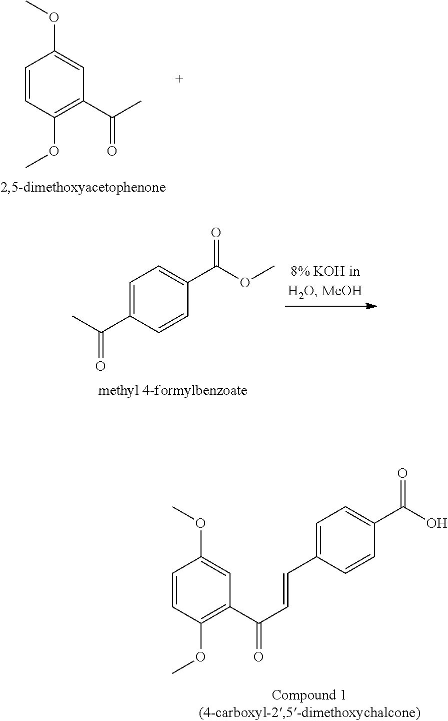

The properties of compound 1 were listed as follows. IR (KBr): 1681, 1597 cm⁻¹. ¹H NMR (CDCl₃): δ 3.82 (3H, s, OCH₃), 3.89 (3H, s, OCH₃), 6.96 (1H, d, J=8.8 Hz, H-3′), 7.06 (1H, dd, J=9.0, 3.2 Hz, H-4′), 7.23 (1H, d, J=3.2 Hz, H-6′), 7.55 (1H, d, J=16.0 Hz, H-α), 7.68 (1H, d, J=16.0 Hz, H-β), 7.69 (2H, dd, J=8.8, 1.6 Hz, H-2 and 6), 8.13 (2H, dd, J=8.8, 1.6 Hz, H-3 and 5). ¹³C NMR (CDCl₃): δ 55.9 (OCH₃), 56.5 (OCH₃), 113.4 (C-6′), 114.4 (C-3′), 119.8 (C-1′ and 4′), 119.8 (C-α), 128.3 (C-2 and 6), 129.3 (C-3 and 5), 130.2 (C-4), 140.4 (C-1), 141.0 (C-β), 152.8 (C-5′), 153.7 (C-2′), 171.0 (—COOH), 191.8 (C═O). ESIMS: m/z 313 [M+H]⁺. Anal. Calcd for C₁₈H₁₆O₅: C, 69.22; H, 5.16. Found: C, 69.10; H, 5.18.

2. Procedure A for synthesis of C-4 ester compounds 2 to 7

To a solution of compound 1 (60 mg, 0.19 mmol) in acetone was added potassium carbonate (K₂CO₃, 52.5 mg, 0.38 mmol) and alkyl halide (0.38 mmol). The reaction mixture was stirred at room temperature for preparing compounds 2 to 4 or refluxed for preparing compounds 5 to 7 overnight. The mixture was concentrated to dryness under reduced pressure, neutralized with 10% HCl solution, and extracted with dichloromethane (CH₂Cl₂). The organic phase was dried over an hydrous sodium sulfate (Na₂SO₄), filtered, and concentrated in vacuo to give the crude product. The crude product was purified by chromatography with EtOAc/n-hexane, and crystallized with acetone/n-hexane (1:4) to afford purified products. Compounds 2 to 7 have the structural formula (Formula I) shown as follows, wherein R1 and R2 substituted groups are depicted in Table 1.

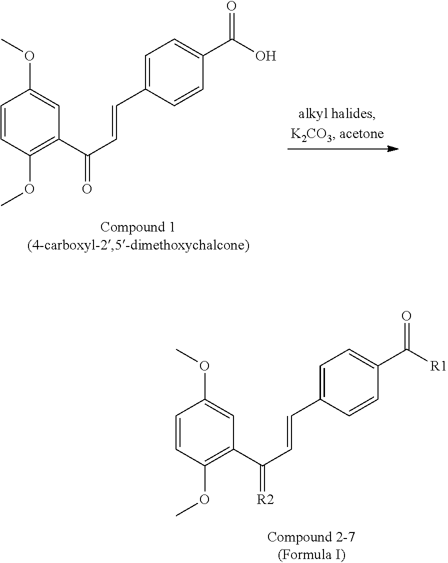

The detailed descriptions for compounds 2 to 7 are provided as follows.

3. Preparation of compound 2 (4-methoxycarbonyl-2′,5′-dimethoxychalcone)

To a solution of compound 1 (60 mg, 0.19 mmol) in acetone was added K₂CO₃ (52.5 mg, 0.38 mmol) and methyl iodide (0.38 mmol). The mixture was treated as procedure A to afford compound 2 (58.2 mg, 92.8%) as a yellow solid, and the properties of compound 2 were listed as follows. IR (KBr): 1721, 1598 cm⁻¹. ¹H NMR (CDCl₃): δ 3.81 (3H, s, OCH₃), 3.87 (3H, s, OCH₃), 3.92 (3H, s, COOCH₃), 6.95 (1H, d, J=8.8 Hz, H-3′), 7.05 (1H, dd, J=8.8, 3.2 Hz, H-4′), 7.21 (1H, d, J=3.2 Hz, H-6′), 7.51 (1H, d, J=16.0 Hz, H-α), 7.64 (2H, d, J=8.0 Hz, H-2 and 6), 7.65 (1H, d, J=16.0 Hz, H-β), 8.05 (2H, d, J=8.4 Hz, H-3 and 5). ¹³C NMR (CDCl₃): δ 52.2 (COOCH₃), 55.8 (OCH₃), 56.4 (OCH₃), 113.4 (C-6′), 114.4 (C-3′), 119.6 (C-1′ and 4′), 119.6 (C-α), 128.1 (C-2 and 6), 128.9 (C-3 and 5), 130.0 (C-4), 139.5 (C-1), 141.2 (C-β), 152.8 (C-5′), 153.6 (C-2′), 166.5 (COOCH₃), 191.8 (C═O). ESIMS: m/z 327 [M+H]⁺. Anal. Calcd for C₁₉H₁₈O₅: C, 69.93; H, 5.56. Found: C, 69.95; H, 5.62.

4. Preparation of compound 3 (4-ethoxycarbonyl-2′,5′-dimethoxychalcone)

To a solution of compound 1 (100 mg, 0.32 mmol) in acetone was added K₂CO₃ (88.5 mg, 0.64 mmol) and ethyl iodide (0.64 mmol). The mixture was treated as procedure A to afford compound 3 (50.0 mg, 45.9%) as a yellow solid, and the properties of compound 3 were listed as follows. IR (KBr): 1715, 1599 cm⁻¹. ¹H NMR (CDCl₃): δ 1.34 (3H, t, J=7.2 Hz, CH₃), 3.81 (3H, s, OCH₃), 3.87 (3H, s, OCH₃), 4.38 (2H, q, J=7.2 Hz, COOCH₂—), 6.95 (1H, d, J=8.8 Hz, H-3′), 7.05 (1H, dd, J=8.8, 3.2 Hz, H-4′), 7.21 (1H, d, J=3.2 Hz, H-6′), 7.51 (1H, d, J=16.0 Hz, H-α), 7.64 (2H, d, J=8.4 Hz, H-2 and 6), 7.65 (1H, d, J=16.0 Hz, H-β), 8.06 (2H, dd, J=8.4, 1.6 Hz, H-3 and 5). ¹³C NMR (CDCl₃): δ 14.3 (CH₃), 55.8 (OCH₃), 56.4 (OCH₃), 61.1 (COOCH₂—), 113.4 (C-6′), 114.4 (C-3′), 119.6 (C-1′ and 4′), 119.6 (C-α), 128.1 (C-2 and 6), 128.8 (C-3 and 5), 130.0 (C-4), 139.4 (C-1), 141.3 (C-β), 152.8 (C-5′), 153.6 (C-2′), 166.0 (COOCH₂—), 191.9 (C═O). ESIMS: m/z 341 [M+H]⁺. Anal. Calcd for C₂₀H₂₀O₅.½H₂O: C, 68.75; H, 6.06. Found: C, 68.71; H, 6.49.

5. Preparation of compound 4 (propoxycarbonyl-2′,5′-dimethoxychalcone)

To a solution of compound 1 (60 mg, 0.19 mmol) in acetone was added K₂CO₃ (52.5 mg, 0.38 mmol) and propyl iodide (0.38 mmol). The mixture was treated as procedure A to afford compound 4 (27.8 mg, 40.8%) as a yellow oil, and the properties of compound 4 were listed as follows. IR (KBr): 1715, 1602 cm⁻¹. ¹H NMR (CDCl₃): δ 1.04 (3H, t, J=7.2 Hz, CH₃), 1.80 (2H, m, CH₂CH₃), 3.81 (3H, s, OCH₃), 3.88 (3H, s, OCH₃), 4.29 (2H, t, J=6.4 Hz, COOCH₂—), 6.95 (11.1, d, J=9.2 Hz, H-3′), 7.05 (1H, dd, J=9.2, 3.2 Hz, H-4′), 7.22 (1H, d, J=3.2 Hz, H-6′), 7.51 (1H, d, J=16.0 Hz, H-α), 7.65 (2H, d, J=8.4 Hz, H-2 and 6), 7.66 (1H, d, J=16.0 Hz, H-β), 8.06 (2H, d, J=8.4 Hz, H-3 and 5). ¹³C NMR (CDCl₃): δ 10.5 (CH₃), 22.1 (CH₂CH₃), 55.8 (OCH₃), 56.4 (OCH₃), 66.7 (COOCH₂—), 113.4 (C-6′), 114.4 (C-3′), 119.7 (C-1′ and 4′), 119.7 (C-α), 128.1 (C-2 and 6), 128.8 (C-3 and 5), 130.0 (C-4), 139.4 (C-1), 141.3 (C-β), 152.8 (C-5′), 153.7 (C-2′), 166.1 (COOCH₂—), 191.9 (C═O). ESIMS: m/z 355 [M+H]⁺. Anal. Calcd for C₂₁H₂₂O₅: C, 71.17; H, 6.26. Found: C, 71.15; H, 6.39.

6. Preparation of compound 5 (4-isopropoxycarbonyl-2′,5′-dimethoxychalcone)

To a solution of compound 1 (60 mg, 0.19 mmol) in acetone was added K₂CO₃ (52.5 mg, 0.38 mmol) and isopropyl iodide (0.38 mmol). The mixture was treated as procedure A to afford compound 5 (58.2 mg, 85.6%) as a yellow oil, and the properties of compound 5 were listed as follows. IR (KBr): 1711, 1603 cm⁻¹. ¹H NMR (CDCl₃): δ 1.37 (6H, d, J=6.0 Hz, CH₃×2), 3.81 (3H, s, OCH₃), 3.87 (3H, s, OCH₃), 5.26 (1H, m, COOCH(CH₃)₂), 6.95 (1H, d, J=9.2 Hz, H-3′), 7.05 (1H, dd, J=8.8, 3.2 Hz, H-4′), 7.21 (1H, d, J=3.2 Hz, H-6′), 7.51 (1H, d, J=16.0 Hz, H-α), 7.64 (2H, d, J=8.8 Hz, H-2 and 6), 7.66 (1H, d, J=16.0 Hz, H-β), 8.05 (2H, d, J=8.4 Hz, H-3 and 5). ¹³C NMR (CDCl₃): δ 21.9 (CH₃×2), 55.9 (OCH₃), 56.5 (OCH₃), 68.6 (COOCH(CH₃)₂), 113.4 (C-6′), 114.4 (C-3′), 119.7 (C-1′ and 4′), 119.7 (C-α), 128.1 (C-2 and 6), 128.8 (C-3 and 5), 130.0 (C-4), 139.3 (C-1), 141.4 (C-β), 152.8 (C-5′), 153.7 (C-2′), 166.1 (COOCH(CH₃)₂), 191.9 (C═O). ESIMS: m/z 355 [M+H]⁺. Anal. Calcd for C₂₁H₂₂O₅.¼H₂O: C, 70.28; H, 6.32. Found: C, 70.64; H, 6.26.

7. Preparation of compound 6 (4-benzyloxycarbonyl-2′,5′-dimethoxychalcone)

To a solution of compound 1 (60 mg, 0.19 mmol) in acetone was added K₂CO₃ (52.5 mg, 0.38 mmol) and benzyl bromide (0.38 mmol). The mixture was treated as procedure A to afford compound 6 (67.7 mg, 87.6%) as a yellow oil, and the properties of compound 6 were listed as follows. IR (KBr): 1717, 1601 cm⁻¹. ¹H NMR (CDCl₃): δ 3.81 (3H, s, OCH₃), 3.87 (3H, s, OCH₃), 5.38 (2H, s, COOCH₂—), 6.95 (1H, d, J=8.8 Hz, H-3′), 7.05 (1H, dd, J=8.8, 3.2 Hz, H-4′), 7.22 (1H, d, J=3.2 Hz, H-6′), 7.35 (5H, m, —C₆H₅), 7.51 (1H, d, J=16.0 Hz, H-α), 7.64 (2H, d, J=8.4 Hz, H-2 and 6), 7.66 (1H, d, J=16.0 Hz, HA, 8.09 (2H, d, J=8.4 Hz, H-3 and 5). ¹³C NMR (CDCl₃): δ 55.9 (OCH₃), 56.5 (OCH₃), 66.9 (COOCH₂—), 113.4 (C-6′), 114.4 (C-3′), 119.7 (C-1′ and 4′), 119.7 (C-α), 128.2-128.3 (tertiary aromatic carbon×5), 128.6 (C-2 and 6), 129.0 (C-3 and 5), 130.2 (C-4), 135.9 (quaternary aromatic carbon×1), 139.6 (C-1), 141.2 (C-β), 152.8 (C-5′), 153.7 (C-2′), 165.8 (COOCH₂—), 191.8 (C═O). ESIMS: m/z 403 [M+H]⁺. Anal. Calcd for C₂₅H₂₂O₅: C, 74.61; H, 5.51. Found: C, 74.63; H, 5.63.

8. Preparation of compound 7 (4-(3-methyl-but-2-enyloxycarbonyl)-2′,5′-dimethoxychalcone)

To a solution of compound 1 (60 mg, 0.19 mmol) in acetone was added K₂CO₃ (52.5 mg, 0.38 mmol) and 1-bromo-3-methyl-2-butene (0.38 mmol). The mixture was treated as procedure A to afford compound 7 (62.5 mg, 85.5%) as a yellow oil, and the properties of compound 7 were listed as follows. IR (KBr): 1714, 1600 cm⁻¹. ¹H NMR (CDCl₃): δ 1.78 (3H, s, CH₃), 1.79 (3H, s, CH₃), 3.81 (3H, s, OCH₃), 3.88 (3H, s, OCH₃), 4.83 (2H, d, J=7.2 Hz, COOCH₂—), 5.47 (1H, m, CH═C(CH₃)₂), 6.95 (1H, d, J=8.8 Hz, H-3′), 7.05 (1H, dd, J=8.8, 3.2 Hz, H-4′), 7.21 (1H, d, J=3.2 Hz, H-6′), 7.51 (1H, d, J=16.0 Hz, H-α), 7.63 (2H, d, J=8.0 Hz, H-2 and 6), 7.65 (1H, d, J=16.0 Hz, H-β), 8.06 (2H, d, J=8.4 Hz, H-3 and 5). ¹³C NMR (CDCl₃): δ 18.1 (CH₃), 25.8 (CH₃), 55.9 (OCH₃), 56.5 (OCH₃), 62.1 (COOCH₂—), 113.4 (C-6′), 114.4 (C-3′), 118.5 (CH═C(CH₃)₂), 119.7 (C-1′ and 4′), 119.7 (C-α), 128.1 (C-2 and 6), 128.8 (C-3 and 5), 130.1 (C-4), 137.1 (CH═C(CH₃)₂), 139.4 (C-1), 141.4 (C-β), 152.8 (C-5′), 153.7 (C-2′), 166.1 (COOCH₂—), 191.9 (C═O). ESIMS: m/z 381 [M+H]⁺. Anal. Calcd for C₂₃H₂₄O₅: C, 72.61; H, 6.36. Found: C, 72.60; H, 6.48.

9. Procedure B for synthesis of C-4 amide compounds 8 to 17

A mixture of compound 1 (156 mg, 0.5 mmol), hydroxybenzotriazole (HOBt, 135.1 mg, 1.0 mmol) and 1-(3-dimethylaminopropyl)-3-ethylcarbodiimide hydrochloride (EDCl, 191.7 mg, 1.0 mmol) was dissolved with CH₂Cl₂, and stirred for 10 minutes. The mixture was added amine (1.0 mmol), and stirred at room temperature for 2 hours. The reaction mixture was concentrated in vacuo to give the crude product. The crude product was purified by chromatography with EtOAc/n-hexane, and crystallized with EtOAc to afford purified products. Compounds 8 to 17 have the structural formula (Formula I) as follows, wherein R1 and R2 substituted groups are depicted in Table 1.

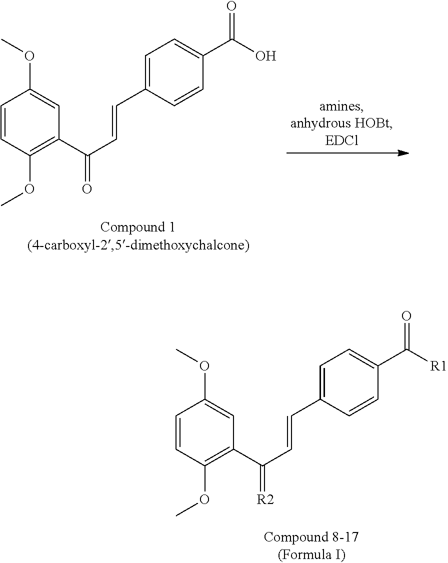

The detailed descriptions for compounds 8 to 17 were provided as follows.

10. Preparation of compound 8 (4-methylcarbamoyl-2′,5′-dimethoxychalcone)

A solution of compound 1 (156 mg, 0.5 mmol), HOBt (135.1 mg, 1.0 mmol) and EDCl (191.7 mg, 1.0 mmol) was dissolved with CH₂Cl₂, and stirred for 10 minutes. The mixture was added methylamine (1.0 mmol) and treated as procedure B to afford compound 8 (82.3 mg, 50.6%) as a yellow solid, and the properties of compound 8 were listed as follows. IR (KBr): 3326, 1652, 1546 cm⁻¹. ¹H NMR (CDCl₃): δ 3.10 (3H, d, J=4.8 Hz, NHCH₃), 3.80 (3H, s, OCH₃), 3.86 (3H, s, OCH₃), 6.39 (1H, br s, CONH—), 6.94 (1H, d, J=9.2 Hz, H-3′), 7.04 (1H, dd, J=9.2, 3.2 Hz, H-4′), 7.19 (1H, d, J=3.2 Hz, H-6′), 7.47 (1H, d, J=16.0 Hz, H-α), 7.61 (2H, d, J=8.4 Hz, H-2 and 6), 7.62 (1H, d, J=16.0 Hz, H-β), 7.78 (2H, d, J=8.4 Hz, H-3 and 5). ¹³C NMR (CDCl₃): δ 26.9 (NHCH₃), 55.8 (OCH₃), 56.5 (OCH₃), 113.4 (C-6′), 114.4 (C-3′), 119.5 (C-1′ and 4′), 119.5 (C-α), 127.4 (C-2 and 6), 128.4 (C-3 and 5), 135.7 (C-4), 138.0 (C-1), 147.5 (C-β), 152.7 (C-5′), 153.6 (C-2′), 167.5 (CONH—), 192.0 (C═O). ESIMS: m/z 326 [M+H]⁺. Anal. Calcd for C₁₉H₁₉NO₄: C, 70.14; H, 5.89; N, 4.31. Found: C, 69.23; H, 5.95; N, 4.21.

11. Preparation of compound 9 (4-ethylcarbamoyl-2′,5′-dimethoxychalcone)

A mixture of compound 1 (156 mg, 0.5 mmol), HOBt (135.1 mg, 1.0 mmol) and EDCl (191.7 mg, 1.0 mmol) was dissolved with CH₂Cl₂, and stirred for 10 minutes. The mixture was added ethylamine (1.0 mmol) and treated as procedure B to afford compound 9 (81.0 mg, 47.8%) as a yellow solid, and the properties of compound 9 were listed as follows. IR (KBr): 3326, 1641, 1544 cm⁻¹. ¹H NMR (CDCl₃): δ 1.25 (3H, t, J=7.2 Hz, CH₃), 3.50 (2H, m, NHCH₂—), 3.80 (3H, s, OCH₃), 3.86 (3H, s, OCH₃), 6.26 (1H, br s, CONH—), 6.94 (1H, d, J=8.8 Hz, H-3′), 7.04 (1H, dd, J=8.8, 3.2 Hz, H-4′), 7.20 (1H, d, J=3.2 Hz, H-6′), 7.47 (1H, d, J=16.0 Hz, H-α), 7.62 (2H, d, J=8.4 Hz, H-2 and 6), 7.63 (1H, d, J=16.0 Hz, H-β), 7.78 (2H, d, J=8.0 Hz, H-3 and 5). ¹³C NMR (CDCl₃): δ 14.8 (CH₃), 35.0 (NHCH₂—), 55.8 (OCH₃), 56.5 (OCH₃), 113.4 (C-6′), 114.4 (C-3′), 119.5 (C-1′ and 4′), 119.5 (C-α), 127.4 (C-2 and 6), 128.4 (C-3 and 5), 135.9 (C-4), 138.0 (C-1), 141.5 (C-β), 152.7 (C-5′), 153.6 (C-2′), 166.7 (CONH—), 192.0 (C═O). EIMS (70 eV) m/z (% rel. int.): 339 (100). Anal. Calcd for C₂₀H₂₁NO₄: C, 70.48; H, 6.24; N, 4.13. Found: C, 69.87; H, 6.28; N, 4.07.

12. Preparation of compound 10 (4-propylcarbamoyl-2′,5′-dimethoxychalcone)

A mixture of compound 1 (156 mg, 0.5 mmol), HOBt (135.1 mg, 1.0 mmol) and EDCl (191.7 mg, 1.0 mmol) was dissolved with CH₂Cl₂, and stirred for 10 minutes. The mixture was added propylamine (1.0 mmol) and treated as procedure B to afford compound 10 (52.9 mg, 30.0%) as a yellow oil, and the properties of compound 10 were listed as follows. IR (KBr): 3329, 1641, 1544 cm⁻¹. ¹H NMR (CDCl₃): δ 0.99 (3H, t, J=7.6 Hz, CH₃), 1.65 (2H, m, CH₂CH₃), 3.43 (2H, m, NHCH₂—), 3.81 (3H, s, OCH₃), 3.87 (3H, s, OCH₃), 6.22 (1H, br s, CONH—), 6.95 (1H, d, J=8.8 Hz, H-3′), 7.02 (1H, dd, J=8.8, 3.2 Hz, H-4′), 7.20 (1H, d, J=3.2 Hz, H-6′), 7.48 (1H, d, J=16.0 Hz, H-α), 7.63 (2H, d, J=8.8 Hz, H-2 and 6), 7.64 (1H, d, J=16.0 Hz, H-β), 7.78 (2H, d, J=8.8 Hz, H-3 and 5). ¹³C NMR (CDCl₃): δ 11.4 (CH₃), 22.9 (CH₂CH₃), 41.8 (NCH₂—), 55.9 (OCH₃), 56.5 (OCH₃), 113.4 (C-6′), 114.4 (C-3′), 119.6 (C-1′ and 4′), 119.6 (C-α), 127.4 (C-2 and 6), 128.4 (C-3 and C-5), 136.0 (C-4), 138.0 (C-1), 141.5 (C-β), 152.7 (C-5′), 153.7 (C-2′), 166.8 (CONH—), 192.0 (C═O). ELMS (70 eV) m/z (% rel. int.): 353 (100). Anal. Calcd for C₂₁H₂₃NO₄.½H₂O: C, 69.58; H, 6.68; N, 3.87. Found: C, 70.60; H, 6.68; N, 3.85.

13. Preparation of compound 11 (4-(2-methylethyl)carbamoyl-2′,5′-dimethoxychalcone)

A mixture of compound 1 (100 mg, 0.32 mmol), HOBt (86.5 mg, 0.64 mmol) and EDCl (122.7 mg, 0.64 mmol) was dissolved with CH₂Cl₂, and stirred for 10 minutes. The mixture was added isopropylamine (1.0 mmol) and treated as procedure B to afford compound 11 (68.7 mg, 60.7%) as a yellow solid, and the properties of compound 11 were listed as follows. IR (KBr): 3320, 1638, 1538 cm⁻¹. ¹H NMR (CDCl₃): δ 1.26 (611, d, J=6.4 Hz, —CH(CH₃)₂), 4.29 (1H, m, NHCH(CH₃)₂), 6.02 (1H, d, J=7.6 Hz, CONH—), 6.94 (1H, d, J=8.8 Hz, H-3′), 7.04 (1H, dd, J=9.0, 3.2 Hz, H-4′), 7.20 (1H, d, J=3.2 Hz, H-6′), 7.47 (1H, d, J=16.0 Hz, H-α), 7.62 (2H, d, J=8.0 Hz, H-2 and 6), 7.63 (1H, d, J=16.0 Hz, H-β), 7.77 (2H, d, J=8.4 Hz, H-3 and 5).

¹³C NMR (CDCl₃): δ 22.8 (CH₃), 29.7 (CH₃), 42.0 (NHCH(CH₃)₂), 55.8 (OCH₃), 56.5 (OCH₃), 113.4 (C-6′), 114.4 (C-3′), 119.5 (C-1′ and 4′), 119.5 (C-α), 127.4 (C-2 and 6), 128.4 (C-3 and 5), 136.1 (C-4), 138.0 (C-1), 141.5 (C-β), 152.7 (C-5′), 153.6 (C-2′), 165.9 (CONH—), 192.0 (C═O). EIMS (70 eV) m/z (% rel. int.): 353 (100). Anal. Calcd for C₂₀H₂₃NO₄: C, 71.37; H, 6.56; N, 3.96. Found: C, 71.57; H, 6.68; N, 3.86.

14. Preparation of compound 12 (4-dimethylcarbamoyl-2′,5′-dimethoxychalcone)

A mixture of compound 1 (100 mg, 0.32 mmol), HOBt (86.5 mg, 0.64 mmol) and EDCl (122.7 mg, 0.64 mmol) was dissolved with CH₂Cl₂, and stirred for 10 minutes. The mixture was added dimethylamine (1.0 mmol) and treated as procedure B to afford compound 12 (53.0 mg, 48.8%) as a yellow oil, and the properties of compound 12 were listed as follows. IR (KBr): 3485, 1633 cm⁻¹. ¹H NMR (CDCl₃): δ 3.09 (6H, m, —N(CH₃)₂), 3.81 (3H, s, —OCH₃), 3.86 (3H, s, —OCH₃), 6.94 (1H, d, J=9.2 Hz, H-3′), 7.03 (1H, dd, J=9.2, 3.2 Hz, H-4′), 7.19 (1H, d, J=3.2 Hz, H-6′), 7.44 (2H, d, J=8.4 Hz, H-2 and 6), 7.45 (1H, d, J=16.0 Hz, H-α), 7.51 (2H, d, J=8.0 Hz, H-3 and 5), 7.63 (1H, d, J=16.0 Hz, H-β). ¹³C NMR (CDCl₃): δ 35.3 and 39.5 (N(CH₃)₂), 55.8 (OCH₃), 56.5 (OCH₃), 113.4 (C-6′), 114.4 (C-3′), 119.5 (C-1′ and 4′), 119.5 (C-α), 127.6 (C-2 and 6), 128.3 (C-3 and 5), 136.3 (C-4), 137.7 (C-1), 141.8 (C-β), 152.7 (C-5′), 153.6 (G-2′), 170.9 (CONH—), 192.0 (C═O). EIMS (70 eV) m/z (% rel. int.): 339 (100). Anal. Calcd for C₂₀H₂₁NO₄: C, 70.48; H, 6.24; N, 4.13. Found: C, 70.10; H, 6.45; N, 4.16.

15. Preparation of compound 13 (4-cyclohexylcarbamoyl-2′,5′-dimethoxychalcone)

A mixture of compound 1 (100 mg, 0.32 mmol), HOBt (86.5 mg, 0.64 mmol) and EDCl (122.7 mg, 0.64 mmol) was dissolved with CH₂Cl₂, and stirred for 10 minutes. The mixture was added cyclohexylamine (1.0 mmol) and treated as procedure B to afford compound 13 (84.0 mg, 66.7%), and the properties of compound 13 were listed as follows. IR (KBr): 3312, 1637, 1540 cm⁻¹. ¹H NMR (CDCl₃): δ 1.24 (2H, m, —CH₂—), 1.44 (2H, m, 1.66 (2H, m, —CH₂—), 1.76 (2H, m, —CH₂—), 2.04 (2H, m, —CH₂—), 3.81 (3H, s, OCH₃), 3.94 (3H, s, OCH₃), 3.98 (1H, m,

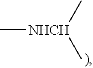

6.00 (1H, d, J=7.6 Hz, CONH—), 6.95 (1H, d, J=8.8 Hz, H-3′), 7.05 (1H, dd, J=8.8, 3.2 Hz, H-4′), 7.21 (1H, d, J=3.2 Hz, H-6′), 7.48 (1H, d, J=16.0 Hz, H-α), 7.63 (2H, d, J=7.6 Hz, H-2 and 6), 7.64 (1H, d, J=16.0 Hz, H-β), 7.77 (2H, d, J=8.4 Hz, H-3 and 5). ¹³C NMR (CDCl₃): δ 24.9 (—CH₂—×2), 25.5 (—CH₂—×1), 33.2 (—CH₂—×2), 48.8

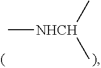

55.9 (OCH₃), 56.5 (OCH₃), 113.4 (C-6′), 114.4 (C-3′), 119.6 (C-1′ and 4′), 119.6 (C-α), 127.4 (C-2 and 6), 128.4 (C-3 and 5), 136.2 (C-4), 138.0 (C-1), 141.5 (C-β), 152.7 (C-5′), 153.7 (C-2′), 165.8 (CONH—), 192.0 (C═O) EIMS (70 eV) m/z (% rel. int.): 393 (100). Anal. Calcd for C₂₄H₂₇NO₄: C, 73.26; H, 6.92; N, 3.56. Found: C, 73.31; H, 7.03; N, 3.55.

16. Preparation of compound 14 (4-diethylcarbamoyl-2′,5′-dimethoxychalcone)

A mixture of compound 1 (100 mg, 0.32 mmol), HOBt (86.5 mg, 0.64 mmol) and EDCl (122.7 mg, 0.64 mmol) was dissolved with CH₂Cl₂, and stirred for 10 minutes. The mixture was added diethylamine (1.0 mmol) as procedure B to afford compound 14 (115.5 mg, 98.2%) as a yellow oil, and the properties of compound 14 were listed as follows. IR (KBr): 3568, 1627 cm⁻¹. ¹H NMR (CDCl₃): δ 1.12 (3H, m, CH₃), 1.24 (3H, m, CH₃), 3.25 (2H, m, NCH₂—), 3.54 (2H, m, NCH₂—), 3.80 (3H, s, OCH₃), 3.86 (3H, s, OCH₃), 6.94 (1H, d, J=8.8 Hz, H-3′), 7.03 (1H, dd, J=9.2, 3.2 Hz, H-4′), 7.19 (1H, d, J=2.8 Hz, H-6′), 7.39 (2H, d, J=8.4 Hz, H-2 and 6), 7.44 (1H, d, J=15.6 Hz, H-α), 7.60 (2H, d, J=8.0 Hz, H-3 and H-5), 7.63 (1H, d, J=15.6 Hz, H-fi).

¹³C NMR (CDCl₃): δ 12.9 (CH₃), 14.2 (CH₃), 39.3 (NCH₂—), 43.2 (NCH₂—), 55.8 (OCH₃), 56.4 (OCH₃), 113.3 (C-6′), 114.4 (C-3′), 119.4 (C-1′ and 4′), 119.4 (C-α), 126.9 (C-2 and 6), 128.4 (C-3 and 5), 135.9 (C-4), 138.7 (C-1), 141.9 (C-β), 152.6 (C-5′), 153.6 (C-2′), 170.5 (—CONH—), 192.1 (C═O). ELMS (70 eV) m/z (% rel. int.): 367 (100). Anal. Calcd for C₂₂H₂₅NO₄.¼H₂O: C, 71.04; H, 6.91; N, 3.77. Found: C, 71.23; H, 6.93; N, 3.65.

17. Preparation of compound 15 (4-tetrahydropyrrolylcarbamoyl-2′,5′-dimethoxychalcone)

A mixture of compound 1 (100 mg, 0.32 mmol), HOBt (86.5 mg, 0.64 mmol) and EDCl (122.7 mg, 0.64 mmol) was dissolved with CH₂Cl₂, and stirred for 10 minutes. The mixture was added pyrrolidine (1.0 mmol) and treated as procedure B to afford compound 15 (80.0 mg, 68.4%) as a yellow oil, and the properties of compound 15 were listed as follows. IR (KBr): 3466, 1619 cm⁻¹. ¹H NMR (CDCl₃): δ 1.89 (4H, m, —CH₂CH₂—), 3.42 (2H, m, NCH₂—), 3.60 (2H, m, NCH₂—), 3.79 (3H, s, OCH₃), 3.85 (3H, s, OCH₃), 6.94 (1H, d, J=8.8 Hz, H-3′), 7.02 (1H, dd, J=8.0, 3.2 Hz, H-4′), 7.18 (1H, d, J=3.2 Hz, H-6′), 7.44 (1H, d, J=16.0 Hz, H-α), 7.52 (2H, d, J=8.0 Hz, H-2 and 6), 7.59 (2H, d, J=8.4 Hz, H-3 and 5), 7.62 (1H, d, J=16.0 Hz, H-β). ¹³C NMR (CDCl₃): δ 24.3 (—CH₂CH₂—), 26.3 (—CH₂CH₂—), 46.2 (NCH₂—), 49.5 (NCH₂—), 55.8 (OCH₃), 56.4 (OCH₃), 113.3 (C-6′), 114.3 (C-3′), 119.4 (C-1′ and 4′), 119.4 (C-α), 127.6 (C-2 and 6), 128.1 (C-3 and 5), 136.5 (C-4), 138.5 (C-1), 141.8 (C-β), 152.6 (C-5′), 153.5 (C-2′), 168.9 (CONH—), 192.0 (C═O). EIMS (70 eV) m/z (% rel. int.): 365 (97), 295 (100). Anal. Calcd for C₂₂H₂₃NO₄.¼H₂O: C, 71.43; H, 6.40; N, 3.79. Found: C, 71.67; H, 6.71; N, 3.6.3

18. Preparation of compound 16 (4-(2-hydroxyethyl)methyl-carbamoyl-2′,5′-dimethoxychalcone)

A mixture of compound 1 (100 mg, 0.32 mmol), HOBt (86.5 mg, 0.64 mmol) and EDCl (122.7 mg, 0.64 mmol) was dissolved with CH₂Cl₂, and stirred for 10 minutes. The mixture was added N-methylethanolamine (1.0 mmol) and treated as procedure B to afford compound 16 (93.6 mg, 79.1%) as a yellow oil, and the properties of compound 16 were listed as follows. IR (KBr): 3403, 1610 cm⁻¹. ¹H NMR (CDCl₃): δ 2.45 (OH), 3.05 (3H, s, NCH₃), 3.10 (1H, m, NCHH—), 3.42 (1H, m, NCHH—), 3.71 (2H, m, CH₂OH), 3.80 (3H, s, OCH₃), 3.85 (3H, s, OCH₃), 6.93 (1H, d, J=9.2 Hz, H-3′), 7.03 (1H, dd, J=8.8, 3.2 Hz, H-4′), 7.19 (1H, d, J=3.2 Hz, H-6′), 7.45 (1H, d, J=16.0 Hz, H-α), 7.46 (2H, d, J=8.0 Hz, H-2 and 6), 7.61 (1H, d, J=9.2 Hz, H-3 and 5), 7.62 (1H, d, J=16.0 Hz, H-β). ¹³C NMR (CDCl₃): δ 38.6 (NCH₃), 51.0 (NCH₂—), 55.8 (OCH₃), 56.4 (OCH₃), 60.6 (—CH₂OH), 113.3 (C-6′), 114.4 (C-3′), 119.5 (C-1′ and 4′), 119.5 (C-α), 127.8 (C-2 and 6), 128.3 (C-3 and 5), 136.7 (C-4), 137.0 (C-1), 141.6 (C-β), 152.7 (C-5′), 153.6 (C-2′), 172.5 (—CON—), 192.0 (C═O). EIMS (70 eV) m/z (% rel. int.): 369 (31), 295 (100). Anal. Calcd for C₂₁H₂₃NO₅: C, 68.28; H, 6.28; N, 3.79. Found: C, 66.78; H, 6.52; N, 3.45.

19. Preparation of compound 17 (N-cyclopropyl-4-[3-cyclopropylimino-3-(2′,5′-dimethoxy-phenyl)-propenyl]-benzamide)

A mixture of compound 1 (100 mg, 0.32 mmol), HOBt (86.5 mg, 0.64 mmol) and EDCl (122.7 mg, 0.64 mmol) was dissolved in CH₂Cl₂, and stirred for 10 minutes. The mixture was added cyclopropylamine (1.0 mmol) and treated as procedure B to afford compound 17 (95.3 mg, 76.2%) as a yellow solid, and the properties of compound 17 were listed as follows. IR (KBr): 3286, 1640, 1536 cm⁻¹. ¹H NMR (CDCl₃): δ 0.60 (2H, m, —CH₂CH₂—), 0.84 (4H, m, —CH₂CH₂), 0.96 (2H, m, —CH₂CH₂—), 2.67 (1H, m, NHCH—), 2.87 (1H, m, ═NHCH—), 6.28 (1H, br s, CONH—), 6.42 (1H, d, J=16.0 Hz, H-α), 6.70 (1H, d, J=2.4 Hz, H-6′), 6.95 (2H, m, H-3′ and 4′), 7.12 (1H, d, J=16.0 Hz, H-β), 7.42 (2H, d, J=8.4 Hz, H-2 and 6), 7.66 (2H, d, J=8.4 Hz, H-3 and H-5). ¹³C NMR (CDCl₃): δ 6.8 (—CH₂—×2), 9.9 (—CH₂—), 10.0 (—CH₂—), 23.1 (—NHCH—), 36.2 (═NCH—), 55.8 (OCH₃), 56.4 (OCH₃), 112.6 (C-6′), 114.5 (C-3′), 115.0 (C-1′ and 4′), 125.7 (C-α), 127.1 (C-2), 133.5 (C-5 and C-6), 133.7 (C-3), 135.6 (C-1), 139.6 (C-β), 150.5 (C-5′), 153.6 (C-2′), 164.6 (C═N—), 168.3 (CONH—). EIMS (70 eV) m/z (% rel. int.): 390 (31). Anal. Calcd for C₂₄H₂₆N₂O₃.¼H₂O: C, 72.98; H, 6.76; N, 7.09. Found: C, 73.01; H, 6.69; N, 7.10.

20. Preparation of compound 18 (3-(3-thiophene)carboxyl-1-(2,5-dimethoxyphenyl)prop-2-en-1-one)

2,5-Dimethoxyacetophenone (450.0 mg, 2.5 mmol) and 5-formyl-2-thiophenecarboxylic acid (390.4 mg, 2.5 mmol) were dissolved in MeOH (50 mL), and added 8% KOH in H₂O (50 mL). The reaction mixture was stirred at room temperature for 72 hours and neutralized with 10% HCl solution (100 mL) to form yellow precipitate. The yellow precipitate was filtered and washed with appropriate amount of water. The crude product was purified by chromatography using EtOAc/n-hexane (2:1), and crystallized with EtOAc to afford compound 18 (469.6 mg, 59.1%) as a yellow solid. The reaction formula of compound 18 (Formula II) was listed as follows.

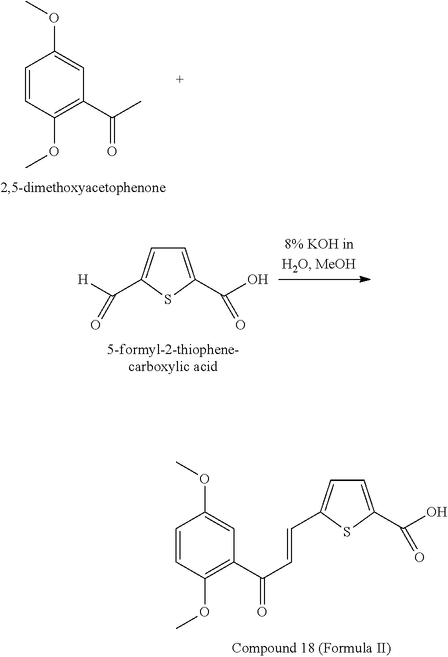

The properties of compound 18 were listed as follows. ¹H NMR (CD₃OD): δ 3.80 (3H, s, OCH₃), 3.90 (3H, s, OCH₃), 7.12 (1H, m, H-3′), 7.14 (1H, m, H-4′), 7.15 (1H, m, H-6′), 7.40 (1H, d, J=15.6 Hz, H-α), 7.42 (1H, d, J=3.2 Hz, H-5), 7.70 (1H, d, 15.6 Hz, H-β), 7.71 (1H, d, J=3.2 Hz, H-4). ¹³C NMR (CD₃OD): δ 56.2 (OCH₃), 56.9 (OCH₃), 114.8 (C-6′), 115.5 (C-3′), 120.7 (C-4′), 129.1 (C-α), 132.8 (C-5), 135.1 (C-4), 135.8 (C-β), 147.5 (C-1), 154.3 (C-5′), 155.2 (C-2′), 193.2 (C═O). ESIMS: m/z 319 [M+H]⁺. Anal. Calcd for C₁₆H₁₄O₅S: C, 60.37; H, 4.43; S, 10.07. Found: C, 60.09; H, 4.42; S, 10.27.

II. Cell Viability/Proliferation Experiments

The cell lines of the present invention were an human urothelial carcinoma cell line (NTUB1), a human prostate cancer cell line (PC3), a human lung adenocarcinoma epithelial cell line (A549) and a SV-40 immortalized human uroepithelial cell line (SV-HUC1). However, the cell lines applied in the present invention includes but not limit in NTUB1, PC3, A549 and SV-HUC1 cells. Other cell lines having the same characteristics or not exactly the same characteristics are able to be used in the present invention. NTUB1, PC3 and A549 cells were maintained in RPMI 1640 and SV-HUC1 cells were maintained in F12 medium supplemented with 10% fetal bovine serum (FBS), 100 unit/mL penicillin-G, 100 μg/mL streptomycin and 2 mM glutamine. The cells were cultured at 37° C. in a humidified atmosphere containing 5% carbon dioxide.

The cytotoxic effect of compounds 1 to 18 of the present invention and cisplatin (positive control) on the cell lines was evaluated using 3-[4,5-dimethylthiazol-2-yl]-2,5-diphenyltetrazolium bromide (MTT) assay. Briefly, the cells were plated at a density of 1800 cells/well in 96-well plates and incubated in the presence of graded concentrations of compounds 1 to 18 and cisplatin at 37° C. for 72 hours. At the end of the culture period, 50 μL of MTT (2 μg mg/mL in PBS) was added to each well and allowed to react for 3 hours. Following centrifugation of plates at 1000 g for 10 minutes, media were removed and 150 μL dimethyl sulfoxide (DMSO) were added to each well. The proportions of surviving cells were determined by absorbance spectrometry at 540 nm. The cell viability was expressed as a percentage to the viable cells of control culture condition. The IC₅₀ value of each group were calculated by the median-effect analysis and presented as mean±standard deviation (SD).

III. Cell Cycle Analysis

DNA content was determined following propidium iodide (PI) staining of cells. Briefly, 6×10⁵ cells were plated and treated with 20 μM cisplatin and various concentrations of compounds 11 and 15 for 24 hours, respectively. These cells were harvested by trypsinization, washed with 1×PBS, and fixed in ice-cold MeOH at −20° C. After overnight incubation, the cells were washed with PBS and incubated with 50 μg/mL propidium iodide and 50 μg/mL RNase A in PBS at room temperature for 30 minutes. The fractions of cells in each phase of cell cycle were analyzed using flow cytometer.

IV. Immuno-Fluorescence Staining

In brief, NTUB 1 cells were seeded onto serum-coated coverslides. The cells were fixed with 2% formaldehyde/PBS for 20 minutes followed by cold methanol permeabilization for 3 minutes. The cells were washed with 1×PBS five minutes twice. The cells were then incubated with α-tubulin primary antibody overnight. The cells were washed with 1×PBS five minutes thrice. Goat anti-rabbit-rhodamine secondary antibody and 1 μg/mL 4′-6-diamidino-2-phenylindole (DAPI) were incubated for 1 hour at room temperature. The slides were washed with 1×PBS, mounted with mounting solution (80% glycerol/PBS) and photographed by microscopy.

V. Western Blot Analysis for α-Tubulin

NTUB1 cells were seeded at a density of 1×10⁶/100-mm² dishes and treated with indicated concentrations of test agents for 6 hours. The cells were then washed with cold PBS and lysed by adding lysis buffer (20 mM Tris-HCl (pH 6.8), 1 mM MgCl₂, 2 mM EGTA (ethylene glycol tetraacetic acid), 20 g/mL aprotinin, 20 g/mL leupeptin, 1 mM phenylmethylsulfonyl fluoride, 1 mM orthovanadate, and 0.5% Nonidet P-40). The cell lysates were centrifuged at 15,000 g for 10 min at 4° C. Soluble and assembled tubulin could be separated in supernatant and pellet fraction, respectively. The supernatants and pellets were revolutionized by the sodium dodecyl sulfate (SDS)-polyacrylamide gel electrophoresis assay. The relative amounts of tubulins transferred on the nitrocellulose membrane were detected by anti-tubulin monoclonal antibody and horseradish peroxidase (HRP)-conjugated secondary antibody. β-Actin was used for control. Detection of immunoreactive signal was accomplished with Western blot chemiluminescent reagent.

### EXPERIMENTAL RESULTS

Please refer to Table 2, which depicts that compound 1 and most of its derivatives showed significantly cytotoxic activities against NTUB1 cells while compound 18 had no significant cytotoxicity against NTUB1 cells. The esterification of C-4-COOH of compound 1 enhanced the cytotoxicity against NTUB1 and PC3 cells. The amidation of C-4-COOH of compound 1 enhanced he cytotoxic effect against the two cancer cell lines while compounds 10, 11 and 15 significantly enhanced the cytotoxicity against NTUB1 and PC3 cells. In Table 2, the compounds possessed of 4-propyl, 4-(2-methylethyl) and 4-tetrahydropyrrolyl carbamoyl groups significantly enhanced the cytotoxicity against NTUB1 and PC3 cell lines.

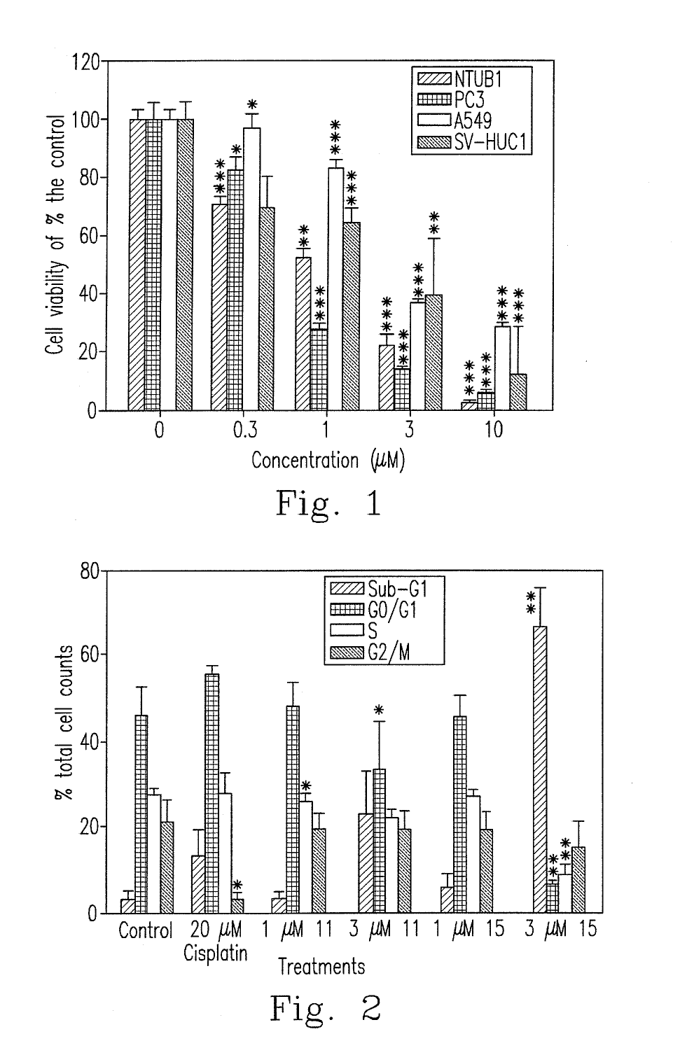

Please refer to FIG. 1, compound 15 showed more potent cytotoxic effect against NTUB1 and PC3 cells than that of A549 cells. It indicated that compound 15 displayed the specific cytotoxicity against cancer cells in the urinary system. In addition, compound 15 exhibited less cytotoxicity against human normal uroepithelial SV-HUC1 cells than those of NTUB1 and PC3 cells lines. It suggested that compound 15 was a good candidate for developing as cancer chemotherapeutic agents for treating cancer related to urinary system.

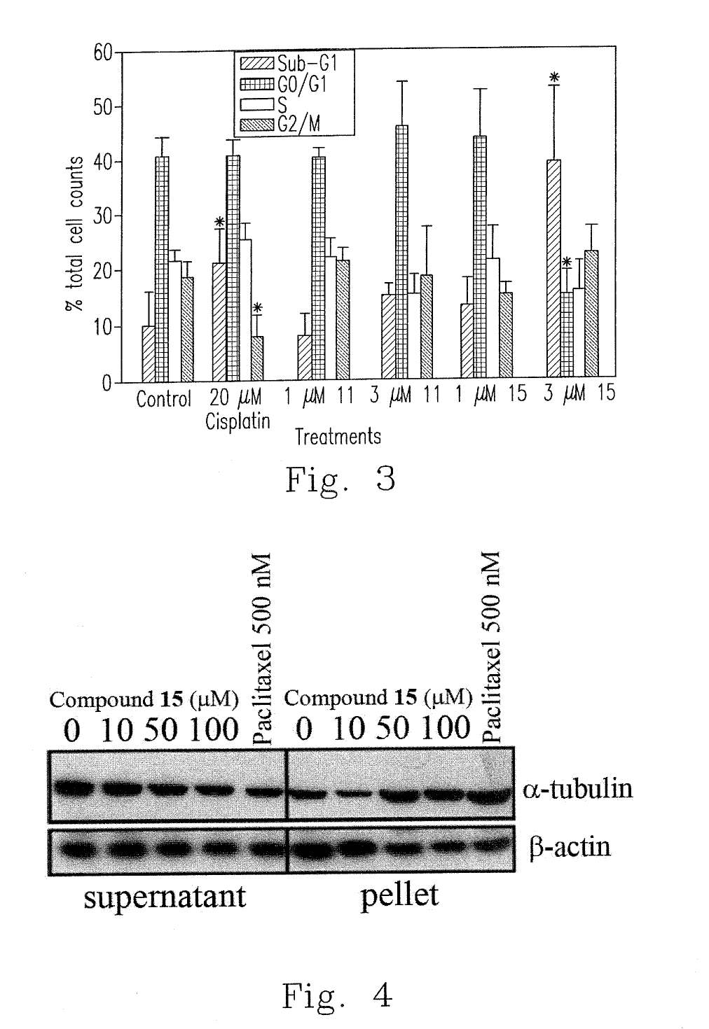

Next, the effect of positive control (cisplatin) and compounds 11 and 15 on cell cycle progression of cancer cells was determined, and the control was indicated to the absence of drug. As shown in FIG. 2, NTUB1 cells treated with 20 μM cisplatin for 24 hours led to an accumulation of cells in G1 and S phases with the concomitant increase of the population of sub-G1 phase. NTUB1 cells were exposed to 1 μM compounds 11 and 15, respectively, for 24 hours induced G1 phase arrest compared to the respective control value, accompanied by an increase in apoptotic cell death, respectively. As shown in FIG. 3, PC3 cells treated with 20 μM cisplatin for 24 hours led to an accumulation of cells in S phase with the concomitant increase of apoptotic cell death. Treatment of PC3 cells with 1 μM compounds 11 and 15, respectively, induced S and G1 phase arrests, respectively, compared to respective control value, accompanied by an increase of apoptotic cells while exposure of PC3 cells to 0.3 μM compounds 11 and 15, respectively, induced G1 and G2/M phase arrests, respectively, compared to the respective control value, accompanied by an increase of the apoptotic cells death. The aforementioned results suggested that compounds 11 and 15 at lower dose revealed same cell cycle arrest in NTUB1 cells while their at higher dose indicated different cell cycle arrest in PC3 cells.

In addition, NTUB1 cells treated with different concentrations of compound 15 for 24 hours showed more cell death and rounding up after treatment (data not shown). Microtubules play an important role in the cell differentiation. If the function of microtubules is blocked by chemicals or compounds (such as paclitaxel), cell differentiation will arrest and be not progressed into the normal pattern of the cell cycle control. It was known from the immuno-fluorescent microscopy that compound 15 induced the microtubule bundle formation in NTUB1 cells and mimicked the effect of paclitaxel. NTUB1 cells treated with 1 μM compound 15 showed that high dosage of compound 15 induced more NTUB1 cells arrested at mitosis and influenced microtubule formation (data not shown). Following compound 15 binding to α-tubulin, it inhibited microtubule dynamic instability, cell cycle G2/M phase transition, mitotic arrest and NTUB1 cell death through apoptosis (as shown in FIG. 2).

Please refer to FIG. 4, NTUB1 cells were treated with 500 nM paclitaxel and more high different concentrations of compound 15 for 6 hours and collected the supernatant and pellet for assays. Paclitaxel, used as the positive control, certainly stabilized the tubulin assembly. α-Tubulin in the supernatant showed a decreased level in a dose-dependent manner after treatment with various concentrations of compound 15 while in the pellet increased in a dose-dependent manner. It suggested that compound 15 could influence tubulin assembly in the molecular action similar to paclitaxel.

Therefore, in the present invention, 4-carboxyl-2′,5′-dimethoxychalcone synthesized from 2,5-dimethoxyacetophenone and methyl 4-formylbenzoate and its 2′,5′-dimethoxylchalcones showed the cytotoxicity on the urinary system-related cancer cells NTUB1 and PC3, wherein compound 15 with a carbamoyl group substituted at C-4 of B ring was found to have strongest cytotoxicity against NTUB1 and PC3 cancer cell lines and showed less cytotoxicity against SV-HUC1 and A549 cells. Thus, it indicated compound 15 specifically inhibited the growth of cancer cells in urinary system. It also indicated that the carbamoyl group substituted at C-4 of the B ring of chalcone moiety may also important structural feature for binding to tubulin. It suggested that compound 15 may be used as microtubule-targeted tubulin-polymerizing agents (MTPAs).

The R1 and R2 substituted groups of the synthesized 2′,5′-dimethoxylchalcone derivatives in the present invention includes but not limit in the substituted groups of compounds 1 to 17. One skilled in the art also can use the derivatives having the substituted groups described in the present invention to synthesize other 2′,5′-dimethoxylchalcone derivatives.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

TABLE 1
Side chains, melting points, yield and molecular formulas of compounds 1 to 18

| Compound | R1 | R2 | Mp (° C.) | Yield (%) | Molecular formula |
|---|---|---|---|---|---|
| 1 | —OH | O | 214-215 | 63.0 | C₁₈H₁₆O₅ |
| 2 | —OCH₃ | O | 118-119 | 92.8 | C₁₉H₁₈O₅ |
| 3 | —OCH₂CH₃ | O | 118-119 | 45.9 | C₂₀H₂₀O₅•1/2H₂O |
| 4 | —OCH₂CH₂CH₃ | O | Oil | 40.8 | C₂₁H₂₂O₅ |
| 5 | —OCH(CH₃)₂ | O | Oil | 85.6 | C₂₁H₂₂O₅•1/4H₂O |
| 6 | —OCH₂C₆H₅ | O | Oil | 87.6 | C₂₅H₂₂O₅ |
| 7 | —OCH₂CH═C(CH₃)₂ | O | Oil | 85.5 | C₂₃H₂₄O₅ |
| 8 | —NHCH₃ | O | 217-218 | 50.6 | C₁₉H₁₉NO₄ |
| 9 | —NHCH₂CH₃ | O | 213-214 | 47.8 | C₂₀H₂₁NO₄ |
| 10 | —NHCH₂CH₂CH₃ | O | Oil | 30.0 | C₂₁H₂₃NO₄•1/2H₂O |
| 11 | —NHCH(CH₃)₂ | O | 227-228 | 60.7 | C₂₀H₂₃NO₄ |
| 12 | —N(CH₃)₂ | O | Oil | 48.8 | C₂₀H₂₁NO₄ |
| 13 | 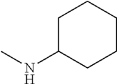 | O | 280-281 | 66.7 | C₂₄H₂₇NO₄ |
| 14 | —N(CH₂CH₃)₂ | O | Oil | 98.2 | C₂₂H₂₅NO₄•1/4H₂O |
| 15 | 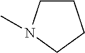 | O | Oil | 68.4 | C₂₂H₂₃NO₄•1/4H₂O |
| 16 | 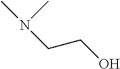 | O | Oil | 79.1 | C₂₁H₂₃NO₅ |
| 17 | 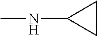 | 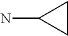 | 282-283 | 76.2 | C₂₄H₂₆N₂O₃•1/4H₂O |
| 18 | | | 237-238 | 59.1 | C₁₆H₁₄O₅S |

TABLE 2
Cytotoxicities of chalcone derivatives against
NTUB1 and PC3 cancer cell lines^{a}

| | NTUB1 | PC3 |
|---|---|---|
| Compound | IC₅₀ (μM) ± SD | IC₅₀ (μM) ± SD |
| Cisplatin | 3.27 ± 0.10 | 4.56 ± 0.76 |
| 1 | 5.70 ± 0.68 | 7.24 ± 0.58 |
| 2 | 3.51 ± 0.45 | 4.85 ± 0.11 |
| 3 | 4.49 ± 0.98 | 5.70 ± 0.43 |
| 4 | 6.01 ± 0.30 | 26.93 ± 0.00 |
| 5 | 4.42 ± 0.50 | 6.74 ± 1.34 |
| 6 | 13.26 ± 2.86 | 17.95 ± 0.50 |
| 7 | 13.41 ± 0.78 | >50 μM |
| 8 | 2.20 ± 0.37 | 2.13 ± 0.21 |
| 9 | 3.76 ± 0.40 | 4.18 ± 0.23 |
| 10 | 2.26 ± 0.31 | 1.50 ± 0.22 |
| 11 | 1.97 ± 0.39 | 1.58 ± 0.08 |
| 12 | 3.89 ± 0.21 | 4.58 ± 1.05 |
| 13 | 2.25 ± 0.57 | 6.30 ± 0.59 |
| 14 | 2.69 ± 0.47 | 3.79 ± 0.56 |
| 15 | 1.26 ± 0.25 | 0.53 ± 0.00 |
| 16 | 4.42 ± 0.15 | 5.75 ± 1.27 |
| 17 | 3.57 ± 0.47 | 4.26 ± 0.81 |
| 18 | >50 μM | n.d |

^{a}Positive control: cisplatin; n.d, not determined, n = 3-5.

What is claimed is:

1. A pharmaceutical composition for treating a cancer comprising a chalcone compound represented by formula I: 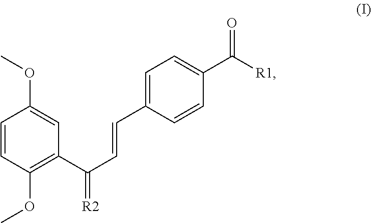
wherein R1 and R2 have one of the following two situations, that (i) R1 is a first substituted group being one selected from a group consisting of a hydroxy group, a C₁-C₆ alkoxy group, an aromatic alkoxy group, a heterocyclic alkoxy group, an alkenyloxy group, an alkylamide group, a C₃-C₆ cycloalkylamide group, a heterocyclic amide group and an —N(CₘH₂ₘ₊₁)—CₙH₂ₙOH group with 1≦m≦6 and when R2 is an oxygen, and (ii) R1 is a cyclopropylamide group when R2 is a cyclopropylimino group.

2. The pharmaceutical composition according to claim 1, wherein the alkenyloxy group is one of an —OCH₂CH═CHCᵣH₂ᵣ₊₁ group with 1≦r≦6 and an —OCH₂CH═C(CₚH₂ₚ₊₁)—C_{q}H_{2q+1} group with 1≦p≦6 and 1≦q≦6.

3. The pharmaceutical composition according to claim 1, wherein the alkylamide group is represented as an —NR—R′ group, and each of R and R′ is a second substituted group being one selected from a group consisting of a hydrogen, a C₁-C₆ saturated hydrocarbon group and a C₁-C₆ unsaturated hydrocarbon group.

4. A pharmaceutical composition for treating a cancer comprising a chalcone compound represented by formula II:

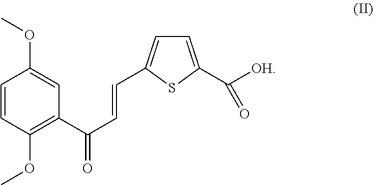

5. The pharmaceutical composition according to claim 4, wherein the chalcone compound is synthesized by reacting a 2,5-dimethoxyacetophenone with a 5-formyl-2-thiophenecarboxylic acid.

6. A preparation method of a pharmaceutical composition for treating a cancer, the method comprising a step of: (a) reacting 2,5-dimethoxyacetophenone with a methyl 4-formylbenzoate to obtain a 4-carboxyl-2′,5′-dimethoxychalcone.

7. The method according to claim 6, wherein the step (a) is performed in an alkaline solution and then is neutralized in a first acidic solution.

8. The method according to claim 7, wherein the alkaline solution is a potassium hydroxide solution, and the first acidic solution is a hydrochloride solution.

9. The method according to claim 6 further comprising a step of (b1) reacting the 4-carboxyl-2′,5′-dimethoxychalcone with an alkyl halide to obtain a first compound represented by formula I: 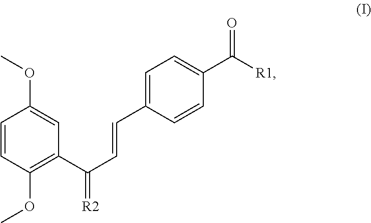
wherein R1 is a first substituted group being one selected from a group consisting of a hydroxy group, a C₁-C₆ alkoxy group, an aromatic alkoxy group, a heterocyclic alkoxy group and an alkenyloxy group when R2 is an oxygen.

10. The method according to claim 9, wherein the alkenyloxy group is one of an —OCH₂CH═CHCᵣH₂ᵣ₊₁ group with 1≦r≦6 and an —OCH₂CH═C(CₚH₂ₚ₊₁)—C_{q}H_{2q+1} group with 1≦p≦6 and 1≦q≦6.

11. The method according to claim 9, wherein the step (b1) further comprises:
(b11) reacting the 4-carboxyl-2′,5′-dimethoxychalcone and the alkyl halide with a potassium carbonate to obtain a first reaction mixture;
(b12) concentrating the first reaction mixture;
(b13) neutralizing the first reaction mixture with a second acidic solution; and
(b 14) extracting the first reaction mixture with a first dichloromethane to obtain the first compound.

12. The method according to claim 11, wherein the second acidic solution is a hydrochloride solution.

13. The method according to claim 6 further comprising a step of: (b2) reacting 4-carboxyl-2′,5′-dimethoxychalcone with an amine to obtain a second compound represented by formula I: 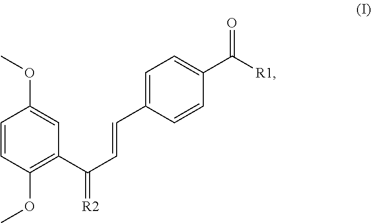
wherein R1 is a second substituted group being one selected from a group consisting of an alkylamide group, a C₃-C₆ cycloalkylamide group, a heterocyclic amide group and a —N(CₘH₂ₘ₊₁)—CₙH₂ₙOH group with 1≦m≦6 and 1≦n≦6 when R2 is an oxygen.

14. The method according to claim 13, wherein the alkylamide group is an —NR—R′ group, and each of R and R′ is a third substituted group being one selected from a group consisting of a hydrogen, a C₁-C₆ saturated hydrocarbon group and a C₁-C₆ unsaturated hydrocarbon group.

15. The method according to claim 13, wherein the step (b2) further comprises:
(b21) dissolving 4-carboxyl-2′,5′-dimethoxychalcone, hydroxybenzotriazole (HOBt) and 1-(3-dimethylaminopropyl)-3-ethylcarbodiimide hydrochloride (EDCl) in a dichloromethane to form a second mixture;
(b22) reacting the second mixture with the amine to obtain a second reaction mixture;
(b23) concentrating the second reaction mixture; and
(b24) crystallizing the second reaction mixture with an ethyl acetate to obtain the second compound.

16. A preparation method of a pharmaceutical composition for treating a cancer, comprising a step of reacting a 2,5-dimethoxyacetophenone with a 5-formyl-2-thiophenecarboxylic acid to obtain a compound represented by formula II:

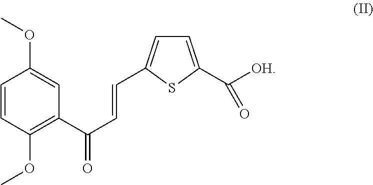

17. The preparation method according to claim 16, wherein the 2,5-dimethoxyacetophenone and the 5-formyl-2-thiophenecarboxylic acid are reacted in an alkaline solution and then neutralized in an acidic solution.

18. The preparation method according to claim 17, wherein the alkaline solution is a potassium hydroxide solution, and the acidic solution is a hydrochloride solution.
